(19) United States
(12) Patent Application Publication
(10) Pub. No.: US 2012/0009226 A1
(43) Pub. Date: Jan. 12, 2012

(54) HIGHLY PURE LAQUINIMOD OR A PHARMACEUTICALLY ACCEPTABLE SALT THEREOF

(75) Inventors: Girish Dixit, Uttar Pradesh (IN); Krishnadatt Sharma, Maharastra (IN); Nitin Sharadchandra Pradhan, Maharashtra (IN)

(73) Assignee: ACTAVIS GROUP PTC EHF, Hafnarfjordur (IS)

(21) Appl. No.: 13/133,994

(22) PCT Filed: Dec. 15, 2009
(86) PCT No.: PCT/IB2009/007946

§ 371 Date: Aug. 8, 2011

(30) Foreign Application Priority Data
Dec. 17, 2008 (IN) 3167/CHE/2008

(51) Int. Cl. A61K 31/4704; A61K 9/14; A61P 25/00; A61P 3/10; A61P 25/28; A61P 29/00; A61P 1/00; A61P 17/06; A61P 11/06; A61P 9/10; C07D 215/56; A61P 19/04

(52) U.S. Cl. 424/400; 546/157; 514/312

(57) ABSTRACT

Provided herein is an impurity of laquinimod, N-ethyl-N-phenyl-1,2-dihydro-4-hydroxy-1-methyl-2-oxoquinoline-3-carboxamide (deschloro laquinimod impurity), and process for preparation and isolation thereof. Provided further herein is a highly pure laquinimod or a pharmaceutically acceptable salt thereof substantially free of deschloro laquinimod impurity, processes for the preparation thereof, and pharmaceutical compositions comprising highly pure laquinimod or a pharmaceutically acceptable salt thereof substantially free of deschloro laquinimod impurity.

### CROSS REFERENCE TO RELATED APPLICATION

This application claims the benefit of priority to Indian provisional application No. 3167/CHE/2008, filed on Dec. 17, 2008, which is incorporated herein by reference in its entirety.

### FIELD OF THE DISCLOSURE

Disclosed herein is an impurity of laquinimod or a pharmaceutically acceptable salt thereof, and processes for the preparation and isolation thereof. Disclosed further herein is a highly pure laquinimod or a pharmaceutically acceptable salt thereof substantially free of impurities, processes for the preparation thereof, and pharmaceutical compositions comprising highly pure laquinimod or a pharmaceutically acceptable salt thereof substantially free of impurities.

### BACKGROUND

U.S. Pat. No. 6,077,851 discloses a variety of quinoline-3-carboxamide derivatives and their salts, processes for their preparation, pharmaceutical compositions comprising the derivatives, and method of use thereof. These compounds are useful for clinical treatment of diseases resulting from autoimmunity such as multiple sclerosis, insulin-dependent diabetes mellitus, systemic lupus erythematosus, rheumatoid arthritis, inflammatory bowel disease and psoriasis and, furthermore, diseases where pathologic inflammation plays a major role, such as asthma, atherosclerosis, stroke and Alzheimer's disease. Of these compounds, N-ethyl-N-phenyl-1,2-dihydro-4-hydroxy-5-chloro-1-methyl-2-oxoquinoline-3-carboxamide is important, since it is well-known as a pharmaceutically active substance under the name of Laquinimod. Laquinimod is a promising immunomodulatory agent and useful for the treatment of multiple sclerosis and its manifestations. Laquinimod is represented by the following structural formula I:

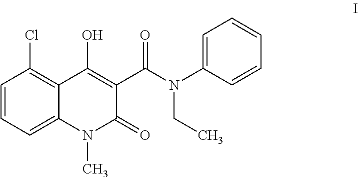

Processes for the preparation of laquinimod and its sodium salt are disclosed in U.S. Pat. Nos. 6,077,851 and 6,875,869.

U.S. Pat. No. 6,077,851 (hereinafter referred to as the '851 patent) describes several synthetic routes for preparing laquinimod. According to a first synthetic process, laquinimod is prepared by the reaction of 1,2-dihydro-4-hydroxy-5-chloro-1-methyl-2-oxo-quinoline-3-carboxylic acid ethyl ester with N-ethylaniline in a suitable solvent such as toluene, xylene and the like, to produce a reaction mass containing laquinimod, followed by distillation of ethanol formed during the reaction and then subjected to usual work up to produce laquinimod, which is then converted into its sodium salt.

According to a second synthetic process as described in the '851 patent, laquinimod is prepared by the reaction of 5-chloro isatoic anhydride with N-ethyl-N-phenylcarbamoyl acetic acid ethyl ester in the presence of methyl iodide and a strong base such as sodium hydride in a suitable solvent such as N,N-dimethylacetamide.

According to a third synthetic process as described in the '851 patent, laquinimod is prepared by the reaction of 1,2-dihydro-4-hydroxy-5-chloro-1-methyl-2-oxo-quinoline-3-carboxylic acid with N-ethylaniline using coupling reagents such as carbodiimides and thionyl chloride in the presence of triethylamine.

Laquinimod obtained by the processes described in the '851 patent is further recrystallized from methanol to produce laquinimod with greater than 95% purity. Laquinimod obtained by the processes described in the above prior art does not have satisfactory purity for pharmaceutical use. Unacceptable amounts of impurities are generally formed along with laquinimod, thus resulting in a poor product yield. In addition, the processes involve the use of additional, explosive and hazardous reagents like sodium hydride, methyl iodide and thionyl chloride. The use of thionyl chloride and sodium hydride is not advisable for scale up operations.

U.S. Pat. No. 6,875,869 (hereinafter referred to as the '869 patent) describes an improved process for the preparation of laquinimod comprising reacting 1,2-dihydro-4-hydroxy-5-chloro-1-methyl-2-oxo-quinoline-3-carboxylic acid methyl ester with N-ethylaniline in the presence of a solvent selected from straight or branched alkanes and cycloalkanes or mixtures thereof with a boiling point between 80° C. and 200° C., specifically n-heptane, n-octane or mixtures thereof.

Two impurities of laquinimod have been disclosed in the '869 patent. These impurities are characterized as 5-chloro-1,2-dihydro-4-hydroxy-1-methyl-2-oxo-quinoline (hereinafter referred to as the Impurity-A) and 5-chloro-1,2-dihydro-4-hydroxy-1-methyl-2-oxo-quinolinecarboxylic acid methyl ester (hereinafter referred to as the Impurity-E), and having the following structural formulae:

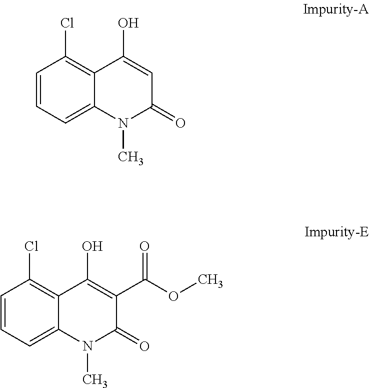

PCT Publication No. WO 2007/047863 discloses a process for recrystallization of laquinimod sodium comprising dissolving laquinimod sodium in water to form an aqueous solution, concentrating the solution to form a concentrated solution, adding a water-miscible anti-solvent to the concentrated solution to form laquinimod sodium crystals, and isolating the laquinimod sodium crystals.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in laquinimod or any active pharmaceutical ingredient (API) are undesirable and might be harmful.

Regulatory authorities worldwide require that drug manufacturers isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product is analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United States Food and Drug Administration guidelines recommend that the amounts of some impurities limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRT”) to identify impurities. The RRT of an impurity is its retention time divided by the retention time of a reference marker.

The person skilled in the art of drug manufacturing research and development understand that a compound in a relatively pure state can be used as a “reference standard”. A reference standard is similar to a reference marker, which is used for qualitative analysis only, but is used to quantify the amount of the compound of the reference standard in an unknown mixture, as well. A reference standard is an “external standard”, when a solution of a known concentration of the reference standard and an unknown mixture are analyzed using the same technique. The amount of the compound in the mixture can be determined by comparing the magnitude of the detector response.

The reference standard can also be used to quantify the amount of another compound in the mixture if a “response factor”, which compensates for differences in the sensitivity of the detector to the two compounds, has been predetermined. For this purpose, the reference standard is added directly to the mixture, and is known as an “internal standard”.

The reference standard can serve as an internal standard when, without the deliberate addition of the reference standard, an unknown mixture contains a detectable amount of the reference standard compound using the technique known as “standard addition”.

In the “standard addition technique”, at least two samples are prepared by adding known and differing amounts of the internal standard. The proportion of the detector response due to the reference standard present in the mixture without the addition can be determined by plotting the detector response against the amount of the reference standard added to each of the samples, and extrapolating the plot to zero concentration of the reference standard.

It is known by those skilled in the art, the management of process impurities is greatly enhanced by understanding their chemical structures and synthetic pathways, and by identifying the parameters that influence the amount of impurities in the final product.

There is a need for highly pure laquinimod or a pharmaceutically acceptable salt thereof substantially free of impurities, as well as processes for preparing thereof.

### SUMMARY

In one aspect, provided herein is an isolated deschloro laquinimod compound, N-ethyl-N-phenyl-1,2-dihydro-4-hydroxy-1-methyl-2-oxoquinoline-3-carboxamide, having the following structural formula I(i):

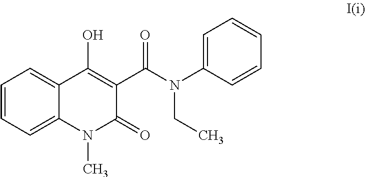

or a pharmaceutically acceptable salt thereof.

In another aspect, provided herein is an impurity of laquinimod, N-ethyl-N-phenyl-1,2-dihydro-4-hydroxy-1-methyl-2-oxoquinoline-3-carboxamide (deschloro laquinimod impurity or Impurity-B), of formula I(i).

In another aspect, encompassed herein is a process for synthesizing and isolating the deschloro laquinimod of formula I(i), also referred to as the “deschloro laquinimod impurity or Impurity-B”, comprising the steps of: a) methylating an isatoic anhydride of formula V(i) with dimethylsulfate in the presence of a base in a first solvent to provide an N-methylisatoic anhydride of formula IV(i); b) reacting the N-methylisatoic anhydride of formula IV(i) with dimethylmalonate in the presence of sodium methoxide in a suitable solvent to provide 1,2-dihydro-4-hydroxy-1-methyl-2-oxo-quinolinecarboxylic acid methyl ester of formula III(i); c) hydrolyzing the ester compound with a suitable acid in a second solvent to provide a 1,2-dihydro-4-hydroxy-1-methyl-2-oxo-quinolinecarboxylic acid of formula II(i); d) condensing the quinolinecarboxylic acid compound of formula II(i) with N-ethylaniline in the presence of a coupling agent in a suitable solvent to provide a reaction mass containing N-ethyl-N-phenyl-1,2-dihydro-4-hydroxy-1-methyl-2-oxoquinoline-3-carboxamide (deschloro laquinimod) of formula 1(i); and e) isolating the deschloro laquinimod from the reaction mass. The synthesis of the deschloro laquinimod impurity is shown in the following scheme:

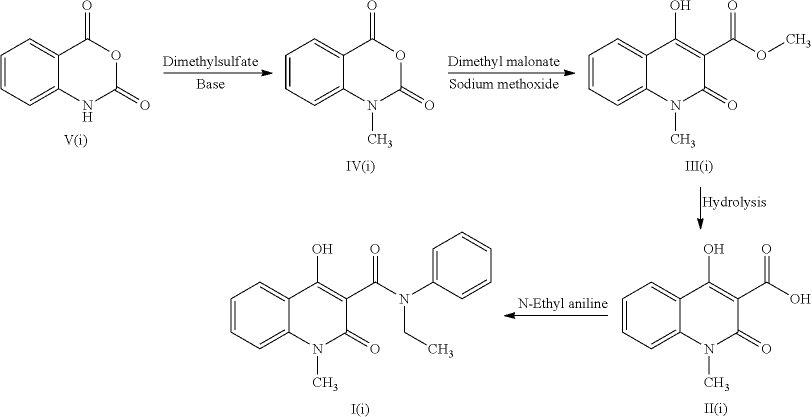

In another aspect, provided herein is a highly pure laquinimod or a pharmaceutically acceptable salt thereof comprising deschloro laquinimod impurity in an amount of about 0.01 area-% to about 0.2 area-%, and specifically in an amount of about 0.01 area-% to about 0.15 area-%, as measured by HPLC.

In another aspect, provided herein is a highly pure laquinimod or a pharmaceutically acceptable salt thereof substantially free of at least one, or more, specifically all, of the deschloro laquinimod (impurity-B), phosphine oxide (impurity-C) and quinolinecarboxylic acid (impurity-D) impurities.

In another aspect, encompassed herein is a process for preparing the highly pure laquinimod or a pharmaceutically acceptable salt thereof having deschloro laquinimod impurity in an amount of about 0.01 area-% to about 0.2 area-%.

In yet another aspect, encompassed herein is a process for preparing the highly pure laquinimod or a pharmaceutically acceptable salt thereof substantially free of at least one, or more, specifically all, of the deschloro laquinimod, phosphine oxide and quinolinecarboxylic acid impurities.

In another aspect, provided herein is a pharmaceutical composition comprising highly pure laquinimod or a pharmaceutically acceptable salt thereof having deschloro laquinimod impurity in an amount of about 0.01 area-% to about 0.2 area-%, and one or more pharmaceutically acceptable excipients.

In still another aspect, provided herein is a pharmaceutical composition comprising highly pure laquinimod or a pharmaceutically acceptable salt thereof having deschloro laquinimod impurity in an amount of about 0.01 area-% to about 0.2 area-% made by the process disclosed herein, and one or more pharmaceutically acceptable excipients.

In still further aspect, encompassed is a process for preparing a pharmaceutical formulation comprising combining highly pure laquinimod or a pharmaceutically acceptable salt thereof having deschloro laquinimod impurity in an amount of about 0.01 area-% to about 0.2 area-% with one or more pharmaceutically acceptable excipients.

In another aspect, the highly pure laquinimod or a pharmaceutically acceptable salt thereof having a deschloro laquinimod impurity in an amount of about 0.01 area-% to about 0.2 area-% disclosed herein for use in the pharmaceutical compositions has a 90 volume-percent of the particles (D₉₀) of less than or equal to about 400 microns, specifically less than or equal to about 300 microns, more specifically less than or equal to about 100 microns, still more specifically less than or equal to about 60 microns, and most specifically less than or equal to about 15 microns.

In another aspect, provided herein is the use of deschloro laquinimod impurity as a reference marker in a qualitative analysis of laquinimod.

In yet another aspect, provided herein is a method of using deschloro laquinimod as a reference standard to analytically quantify the purity of laquinimod sodium.

In yet a further aspect, provided herein is a method for the quantification of the purity of laquinimod sodium, comprising the use of deschloro laquinimod as a reference standard, where reference standard may be either external or internal standard.

### DETAILED DESCRIPTION

According to one aspect, there is provided a deschloro laquinimod compound, N-ethyl-N-phenyl-1,2-dihydro-4-hydroxy-1-methyl-2-oxoquinoline-3-carboxamide, having the following structural formula I(i):

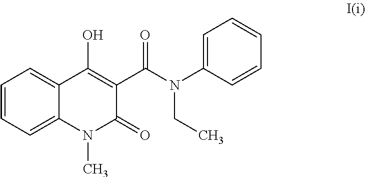

or a pharmaceutically acceptable salt thereof.

The pharmaceutically acceptable salts of deschloro laquinimod can be derived from alkali or alkaline earth metals include the sodium, calcium, potassium and magnesium, and a more specific salt being deschloro laquinimod sodium.

According to another aspect, there is provided an impurity of laquinimod, the deschloro laquinimod impurity (Impurity-B), N-ethyl-N-phenyl-1,2-dihydro-4-hydroxy-1-methyl-2-oxoquinoline-3-carboxamide, of formula I(i).

The deschloro laquinimod impurity has been identified, isolated and synthesized. The deschloro laquinimod impurity was detected and resolved from laquinimod by HPLC with an RRt of 0.6. The structure of the compound of formula I(i) was deduced with the aid of ¹H, ¹³C NMR and IR spectroscopy and FAB mass spectrometry. The parent ion at 322 is consistent with the assigned structure.

The deschloro laquinimod disclosed herein is characterized by data selected from a ¹H NMR (400MHz, DMSO-d6) δ (ppm): 1.03-1.17 (t), 3.70-3.78 (m), 7.01-7.34 (m), 7.48-7.55 (m), 7.76-7.95 (m), 11.16 (brs); MS: EI⁺ m/z (MH+): 322; and IR spectra on KBr having absorption bands at about 754, 1092, 1328, 1383, 1498, 1583, 1648, 2981 and 3431 cm⁻¹.

According to another aspect, there is provided an isolated deschloro laquinimod impurity. Deschloro laquinimod formed during the synthesis of laquinimod or a pharmaceutically acceptable salt thereof can be isolated by subjecting the laquinimod or a pharmaceutically acceptable salt thereof that contains the deschloro laquinimod impurity to column chromatography. The column chromatography comprises using a silica gel, as a stationary phase, and a gradient of eluents that remove deschloro laquinimod impurity from the column on which it adsorbed.

In one embodiment, the deschloro laquinimod of formula I(i) is prepared as per the process exemplified in the Example 1 as disclosed herein.

In addition to the deschloro impurity, there are two other impurities, whose presence was observed in laquinimod and they have not been reported in the prior art. The details of these two impurities of laquinimod are as follows:
i) Impurity-C: triphenylphosphine oxide (hereinafter referred to as the ‘phosphine oxide impurity’), which has the following structural formula:

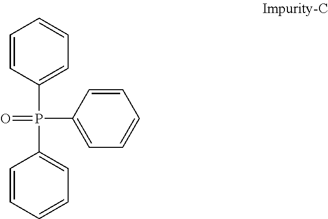

and it is detected and resolved from laquinimod by HPLC with an RRt of 0.92;
ii) Impurity-D: 5-chloro-1,2-dihydro-4-hydroxy-1-methyl-2-oxo-quinolinecarboxylic acid, which has the following structural formula:

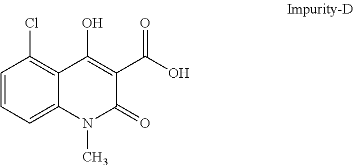

and it is detected and resolved from laquinimod by HPLC with an RRt of 1.12 quinolinecarboxylic acid impurity).

The present inventors have surprisingly found that the deschloro laquinimod impurity is formed as an impurity in the synthesis of laquinimod due to the contamination of the key starting material 5-chloroisatoic anhydride with isatoic anhydride. The triphenylphosphine oxide is formed as an impurity in the synthesis of laquinimod due to the degradation of dichlorotriphenylphosphorane used as a coupling agent in the reaction between 5-chloro-1,2-dihydro-4-hydroxy-1-methyl-2-oxo-quinolinecarboxylic acid and N-ethylaniline.

Regarding the specific RRt values of impurities disclosed herein, it is well known to a person skilled in the art that the RRt values may vary from sample to sample due to, inter alia, instrument errors (both instrument to instrument variation and the calibration of an individual instrument) and differences in sample preparation. Thus, it has been generally accepted by those skilled in the art that independent measurement of an identical RRt value can differ by amounts of up to ±0.01.

Thus there is a need for a method for determining the level of impurities in laquinimod samples and removing the impurities.

Extensive experimentation was carried out by the present inventors to reduce the level of the deschloro laquinimod, phosphine oxide and quinolinecarboxylic acid impurities in laquinimod. As a result, it has been found that the deschloro laquinimod, phosphine oxide and quinolinecarboxylic acid impurities formed in the preparation of the laquinimod can be reduced or completely removed by the processes disclosed herein.

According to another aspect, there is provided a highly pure laquinimod or a pharmaceutically acceptable salt thereof comprising deschloro laquinimod impurity (impurity-B) of formula I(i) in an amount of about 0.01 area-% to about 0.2 area-% as measured by HPLC. Specifically, the laquinimod, as disclosed herein, contains about 0.01 area-% to about 0.15 area-%, more specifically about 0.01 area-% to about 0.1 area-%, of the deschloro laquinimod impurity.

In one embodiment, the highly pure laquinimod or a pharmaceutically acceptable salt thereof disclosed herein is substantially free of at least one, or more, specifically all, of the deschloro laquinimod, phosphine oxide and quinolinecarboxylic acid impurities.

According to another aspect, there is provided a highly pure laquinimod or a pharmaceutically acceptable salt thereof disclosed herein is substantially free of at least one, or more, specifically all, of the deschloro laquinimod, phosphine oxide and quinolinecarboxylic acid impurities.

As used herein, “highly pure laquinimod or a pharmaceutically acceptable salt thereof. disclosed herein is substantially free of at least one, or more, of the deschloro laquinimod, phosphine oxide and quinolinecarboxylic acid impurities” refers to laquinimod or a pharmaceutically acceptable salt thereof comprising one, or more, of the deschloro laquinimod, phosphine oxide and quinolinecarboxylic acid impurities, each one, in an amount of less than about 0.2 area-% as measured by HPLC. Specifically, the laquinimod, as disclosed herein, contains less than about 0.15 area-%, more specifically less than about 0.05 area-%, still more specifically less than about 0.02 area-% of one, or more, of the deschloro laquinimod, phosphine oxide and quinolinecarboxylic acid impurities, and most specifically is essentially free of one, or more, of the deschloro laquinimod, phosphine oxide and quinolinecarboxylic acid impurities.

In one embodiment, the highly pure laquinimod or a pharmaceutically acceptable salt thereof disclosed herein comprises one, or more, of the deschloro laquinimod, phosphine oxide and quinolinecarboxylic acid impurities each in an amount of about 0.01 area-% to about 0.15 area-%, specifically in an amount of about 0.01 area-% to about 0.05 area-%, as measured by HPLC.

In another embodiment, the highly pure laquinimod or a pharmaceutically acceptable salt thereof disclosed herein has a total purity of greater than about 98%, specifically greater than about 99%, more specifically greater than about 99.9%, and most specifically greater than about 99.95% as measured by HPLC. For example, the purity of the highly pure laquinimod or a pharmaceutically acceptable salt thereof is about 98% to about 99.95%, or about 99.5% to about 99.99%.

In another embodiment, the highly pure laquinimod or a pharmaceutically acceptable salt thereof disclosed herein is essentially free of one, or more, of the deschloro laquinimod, phosphine oxide and quinolinecarboxylic acid impurities.

The term “laquinimod or a pharmaceutically acceptable salt thereof essentially free of at least one, or more, of the deschloro laquinimod, phosphine oxide and quinolinecarboxylic acid impurities” refers to laquinimod or a pharmaceutically acceptable salt thereof contains a non-detectable amount of one, or more, of the deschloro laquinimod, phosphine oxide and quinolinecarboxylic acid impurities as measured by HPLC.

According to another aspect, there is provided a process for preparing highly pure laquinimod of formula I or a pharmaceutically acceptable salt thereof comprising deschloro laquinimod impurity (impurity-B) of formula I(i) in an amount of about 0.01 area-% to about 0.2 area-%, comprising:
a) methylating a 5-chloroisatoic anhydride of formula V:

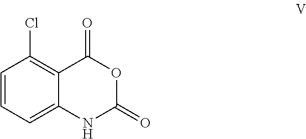

containing isatoic anhydride impurity of formula V(i) in an amount of less than about 1.5 area-% (measured by HPLC), with dimethylsulfate in the presence of a base in a first solvent to provide a 5-chloro-N-methylisatoic anhydride of formula IV:

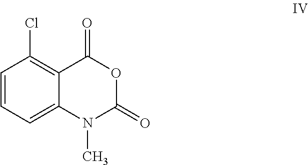

containing N-methylisatoic anhydride impurity of formula IV(i) in an amount of less than about 0.8 area-%;
b) reacting the compound obtained in step-(a) with dimethyl malonate in the presence of sodium methoxide in a second solvent to provide 5-chloro-1,2-dihydro-4-hydroxy-1-methyl-2-oxo-quinolinecarboxylic acid methyl ester of formula III:

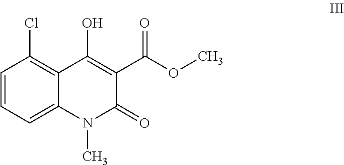

containing 1,2-dihydro-4-hydroxy-1-methyl-2-oxo-quinolinecarboxylic acid methyl ester impurity of formula III(i) in an amount of less than about 0.5 area-%;
c) hydrolyzing the ester compound obtained in step-(b) by treatment with hydrochloric acid in the presence of acetic acid to provide 5-chloro-1,2-dihydro-4-hydroxy-1-methyl-2-oxo-quinoline carboxylic acid of formula II:

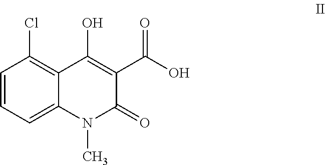

containing 1,2-dihydro-4-hydroxy-1-methyl-2-oxo-quinoline carboxylic acid impurity of formula II(i) in an amount of about 0.01 area-% to about 0.2 area-%;
d) condensing the quinolinecarboxylic acid compound obtained in step-(c) with N-ethylaniline in the presence of a coupling agent in a third solvent to provide a reaction mass containing N-ethyl-N-phenyl-1,2-dihydro-4-hydroxy-5-chloro-1-methyl-2-oxoquinoline-3-carboxamide (laquinimod) of formula I containing N-ethyl-N-phenyl-1,2-dihydro-4-hydroxy-1-methyl-2-oxoquinoline-3-carboxamide impurity (deschloro laquinimod impurity) of formula I(i); and
e) isolating and/or recovering highly pure laquinimod of formula I containing deschloro laquinimod impurity (impurity-B) of formula I(i) in an amount of about 0.01 area-% to about 0.2 area-% as measured by HPLC from the reaction mass obtained in step-(d), and optionally converting the laquinimod obtained into a pharmaceutically acceptable salt thereof.

In one embodiment, the first and second solvents used in steps-(a) and (b) are, each independently, selected from the group consisting of N,N-dimethylformamide, N,N-dimethylacetamide, dimethylsulfoxide, tetrahydrofuran, dioxane, and mixtures thereof. The term solvent also includes mixtures of solvents. A most specific solvent is N,N-dimethylformamide.

In another embodiment, the base used in step-(a) is an organic or inorganic base. Specific organic bases are triethylamine, tributylamine, diisopropylethylamine, diethylamine, tert-butyl amine, N-methylmorpholine, pyridine, 4-(N,N-dimethylamino)pyridine, and mixtures thereof. Exemplary inorganic bases include, but are not limited to, ammonia; hydroxides, alkoxides, carbonates and bicarbonates of alkali or alkaline earth metals. Specific inorganic bases are ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide, potassium tert-butoxide, and mixtures thereof; and more specifically sodium hydroxide, potassium hydroxide, sodium carbonate and potassium carbonate.

In one embodiment, the reaction in step-(a) is carried out at a temperature of about 0° C. to about 100° C. for at least 30 minutes, specifically at a temperature of about 0° C. to about 50° C. for about 45 minutes to about 5 hours, and most specifically at a temperature of about 0° C. to about 35° C. for about 1 hour to about 4 hours.

In another embodiment, the reaction mass containing the compound of formula IV obtained in step-(a) is treated with an acid, preferably hydrochloric acid, and the resulting reaction mass may be subjected to usual work up such as a washing, a filtration, an extraction, an evaporation, or a combination thereof, followed by isolation as solid from a suitable solvent by methods such as cooling, seeding, partial removal of the solvent from the solution, by adding an anti-solvent to the solution, evaporation, vacuum drying, spray drying, freeze drying, or a combination thereof. The reaction mass may be used directly in the next step to produce the compound of formula III, or the compound of formula IV may be isolated and then used in the next step.

In one embodiment, the reaction in step-(b) is carried out at a temperature of below about 110° C. for at least 15 minutes, specifically at a temperature of about 50° C. to about 100° C. for about 30 minutes to about 8 hours, and most specifically at a temperature of about 70° C. to about 90° C. for about 1 hour to about 5 hours. The reaction is advantageously conducted under an inert atmosphere such as nitrogen.

The reaction mass containing the compound of formula III obtained in step-(b) is preferably cooled to below 35° C., water is added to the resulting mass followed by treatment with an acid, preferably hydrochloric acid, and the resulting solid is filtered and subjected to usual work up methods as described above.

The hydrochloric acid used in step-(c) may be in the form of concentrated hydrochloric acid, aqueous hydrochloric acid, in the form of hydrogen chloride gas, or hydrogen chloride dissolved in an organic solvent. Specifically, the hydrochloric acid is used in the form of hydrogen chloride gas. The organic solvent used for dissolving hydrogen chloride gas or hydrogen chloride is selected from the group consisting of ethanol, methanol, isopropyl alcohol, ethyl acetate, diethyl ether, dimethyl ether, acetone, and mixtures thereof.

In one embodiment, the reaction in step-(c) is carried out at a temperature of below about 110° C. for at least 30 minutes, specifically at a temperature of about 75° C. to about 100° C. for about 1 hour to about 8 hours, and most specifically at a temperature of about 80° C. to about 90° C. for about 2 hours to about 5 hours.

In another embodiment, the reaction mass containing the compound of formula II obtained in step-(c) is cooled at a temperature of about 50° C. to about 65° C. followed by the addition of an alcoholic solvent under stirring at about 50° C. to about 65° C., the resulting suspension is cooled and stirred at a temperature of below about 35° C., and the resulting solid is filtered and subjected to usual work up methods as described above. Exemplary alcohol solvents include, but are not limited to, methanol, ethanol, isopropyl alcohol, n-propanol, tert-butanol, n-butanol, amyl alcohol, isoamyl alcohol, and mixtures thereof. Specific alcohol solvents are methanol, ethanol, isopropyl alcohol and mixtures thereof, and most specifically methanol.

In one embodiment, the coupling agent used in step-(d) is selected from the group consisting of carbonyl compounds such as N,N′-carbonyldiimidazole (CDI); and carbodiimides such as N,N′-diethylcarbodiimide, N,N′-dipropylcarbodiimide, N,N′-diisopropylcarbodiimide, N,N′-dicyclohexylcarbodiimide, N-ethyl-N′-(3-dimethylaminopropyl)-carbodiimide; acid halides such as thionyl chloride; and phosphorous compounds such as dichlorotriphenyl phosphorane, phosphorous oxychloride, O-benzotriazole-N,N,N′,N′-tetramethyl-uronium-hexafluoro-phosphate (HBTU); and combinations comprising one or more of the foregoing coupling agents. A specific coupling agent is dichlorotriphenyl phosphorane.

Exemplary third solvents used in step-(d) include, but are not limited to, a hydrocarbon, a chlorinated hydrocarbon, a ketone, a polar aprotic solvent, an ether, a nitrile, an ester, and mixtures thereof.

Specifically, the third solvent is selected from the group consisting of pentane, hexane, heptane, cyclohexane, toluene, xylene, methylene chloride, ethyl dichloride, chloroform, carbon tetrachloride, acetone, methyl isobutyl ketone, N,N-dimethylformamide, N,N-dimethylacetamide, dimethylsulfoxide, diisopropyl ether, methyl tert-butyl ether, tetrahydrofuran, dioxane, acetonitrile, ethyl acetate, isopropyl acetate, and mixtures thereof; and most specifically, the third solvent is selected from the group consisting of hexane, heptane, cyclohexane, toluene, methylene chloride, and mixtures thereof.

The reaction temperature and time period for coupling reaction generally depends on the starting compounds, the coupling agent, the base and the solvent employed in the reaction.

In one embodiment, the coupling reaction is carried out at a temperature of 0° C. to the reflux temperature of the solvent used for at least 20 minutes, specifically at a temperature of about 25° C. to the reflux temperature of the solvent for about 1 hour to about 20 hours, and most specifically at the reflux temperature of the solvent for about 3 hours to about 7 hours. The reaction is advantageously carried out under an inert atmosphere such as nitrogen.

As used herein, “reflux temperature” means the temperature at which the solvent or solvent system refluxes or boils at atmospheric pressure.

In another embodiment, the reaction in step-(d) is carried out in the presence or absence of a base. The base can be an organic or inorganic base selected from the group as described above. A most specific base is diisopropylethylamine.

The isolation of highly pure laquinimod in step-(e) is carried out by forcible or spontaneous crystallization by using a suitable solvent.

Spontaneous crystallization refers to crystallization without the help of an external aid such as seeding, cooling etc., and forcible crystallization refers to crystallization with the help of an external aid.

Forcible crystallization is initiated by a method such as cooling, seeding, partial removal of the solvent from the solution, by combining an anti-solvent with the solution, or a combination thereof.

In one embodiment, the crystallization is carried out by cooling the solution while stirring at a temperature of below 30° C. for at least 15 minutes, specifically at about 0° C. to about 30° C. for about 30 minutes to about 20 hours, and more specifically at about 0° C. to about 20° C. for about 1 hour to about 5 hours.

The recovery of highly pure laquinimod in step-(e) is accomplished by techniques such as filtration, filtration under vacuum, decantation, centrifugation, or a combination thereof. In one embodiment, the laquinimod is recovered by filtration employing a filtration media of, for example, a silica gel or celite.

In another embodiment, the reaction mass obtained in step-(d) is cooled at a temperature of below about 35° C. followed by the addition of water and stirring to separate the layers, and the resulting organic layer may be subjected to usual work methods as described above. The resulting organic layer is suspended in a mixture of methylene chloride and ethyl acetate, the resulting suspension is stirred at reflux temperature followed by cooling the suspension and recovering laquinimod of formula I having deschloro laquinimod impurity (impurity-B) of formula I(i) in an amount of about 0.01 area-% to about 0.2 area-% as measured by HPLC.

Pharmaceutically acceptable salts of laquinimod in step-(e) can be prepared in high purity by using the highly pure laquinimod containing the deschloro laquinimod impurity in an amount of about 0.01 area-% to about 0.2 area-% obtained by the method disclosed herein, by known methods.

Specific pharmaceutically acceptable salts of laquinimod are obtained from alkali or alkaline earth metals include the sodium, calcium, potassium and magnesium, and a most specific salt is laquinimod sodium.

The highly pure laquinimod or a pharmaceutically acceptable salt thereof obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

According to another aspect, there is provided a process for the preparation of highly pure laquinimod or a pharmaceutically acceptable salt thereof substantially free of at least one, or more, of the deschloro laquinimod, phosphine oxide and quinolinecarboxylic acid impurities, comprising:
a) providing a suspension of crude laquinimod in a solvent medium comprising an ester solvent and a chlorinated hydrocarbon solvent;
b) optionally, cooling the suspension obtained in step-(a); and
c) recovering highly pure laquinimod substantially free of impurities from the suspension and optionally converting the highly pure laquinimod obtained into a pharmaceutically acceptable salt thereof.

Exemplary halogenated hydrocarbon solvents used in step-(a) include, but are not limited to, methylene chloride, 1,2-dichloroethane, chloroform, carbon tetrachloride, and mixtures thereof. A specific halogenated hydrocarbon solvent is methylene chloride. Exemplary ester solvents used in step-(a) include, but are not limited to, C₂ to C₆ alkyl acetates such as methyl acetate, ethyl acetate, n-propyl acetate, isopropyl acetate, n-butyl acetate, isobutyl acetate, tert-butyl acetate, ethyl formate, and mixtures thereof. Specific ester solvents are ethyl acetate, isopropyl acetate, and mixtures thereof.

Step-(a) of providing a suspension of crude laquinimod includes suspending crude laquinimod in the solvent medium while stirring at below reflux temperature of the solvent medium used. In one embodiment, the suspension is stirred at a temperature of about 30° C. to about 100° C. for at least 15 minutes and more specifically at about 40° C. to about 80° C. for about 30 minutes to about 5 hours.

In one embodiment, the suspension in step-(a) is prepared by reacting 1,2-dichloro-4-hydroxy-5-chloro-1-methyl-2-oxo-quinoline-3-carboxylic acid with N-ethyl aniline in the presence of a suitable coupling agent, optionally in the presence of a base, in a suitable solvent to produce a reaction mass containing crude laquinimod acid, followed by usual work up such as washings, extractions, evaporations, or a combination thereof. In one embodiment, the work-up includes suspending the resulting crude laquinimod in the solvent medium while stirring at below reflux temperature of the solvent medium, specifically at a temperature of about 30° C. to about 100° C., and more specifically at about 40° C. to about 80° C. for

Alternatively, the suspension in step-(a) is prepared by treating a pharmaceutically acceptable base addition salt of laquinimod with an acid to liberate laquinimod acid and suspending the laquinimod acid in the solvent medium while stirring at below reflux temperature of the solvent medium used.

The treatment of the pharmaceutically acceptable salt of laquinimod with acid is carried out in any solvent and the selection of solvent is not critical. A wide variety of solvents such as chlorinated solvents, hydrocarbon solvents, ether solvents, alcoholic solvents, ketone solvents, ester solvents etc., can be used.

The acid can be inorganic or organic acid. Specific acids are hydrochloric acid, hydrobromic acid, sulfuric acid, nitric acid, acetic acid, propionic acid, phosphoric acid, succinic acid, maleic acid, fumaric acid, citric acid, glutaric acid, citraconic acid, glutaconic acid, tartaric acid, malic acid, ascorbic acid, and more specifically hydrochloric acid.

The suspension in step-(b) is specifically cooled at a temperature of below 30° C. under stirring for at least 15 minutes, and more specifically at a temperature of about 10° C. to about 30° C. for about 30 minutes to 2 hours.

The recovering in step-(c) is carried out by methods such as filtration, filtration under vacuum, decantation, centrifugation, or a combination thereof. In one embodiment, the laquinimod is recovered by filtration employing a filtration media of, for example, a silica gel or celite.

Pharmaceutically acceptable salts of laquinimod in step-(c) can be prepared in high purity by using the highly pure laquinimod substantially free of impurities obtained by the method disclosed herein, by known methods.

The highly pure laquinimod or a pharmaceutically acceptable salt thereof obtained by the above process may be further dried by the methods described hereinabove.

According to another aspect, there is provided a process for synthesizing and isolating the deschloro laquinimod of formula I(i) or a pharmaceutically acceptable salt thereof, comprising:
a) methylating an isatoic anhydride of formula V(i):

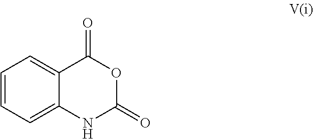

with dimethylsulfate in the presence of a base in a first solvent to provide a N-methylisatoic anhydride of formula IV(i):

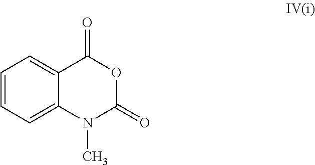

b) reacting the N-methylisatoic anhydride with dimethyl malonate in the presence of sodium methoxide in a second solvent to provide a 1,2-dihydro-4-hydroxy-1-methyl-2-oxo-quinolinecarboxylic acid methyl ester of formula III(i):)

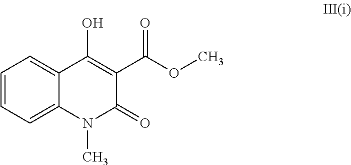

c) hydrolyzing the ester compound of formula III(i) with an acid in a third solvent to provide a 1,2-dihydro-4-hydroxy-1-methyl-2-oxo-quinolinecarboxylic acid of formula II(i):

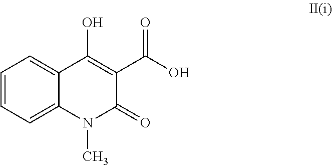

d) condensing the quinolinecarboxylic acid compound of formula II(i) with N-ethylaniline in the presence of a coupling agent in a fourth solvent to provide a reaction mass containing N-ethyl-N-phenyl-1,2-dihydro-4-hydroxy-1-methyl-2-oxoquinoline-3-carboxamide (deschloro laquinimod) of formula I(i); and
e) isolating and/or recovering the deschloro laquinimod from the reaction mass obtained in step-(d) and optionally converting the deschloro laquinimod obtained into a pharmaceutically acceptable salt thereof.

In one embodiment, the first and second solvents used in steps-(a) and (b) are, each independently, selected from the group consisting of N,N-dimethylformamide, N,N-dimethylacetamide, dimethylsulfoxide, tetrahydrofuran, dioxane, and mixtures thereof. The term solvent also includes mixtures of solvents. A most specific solvent is N,N-dimethylformamide.

The base used in step-(a) is an organic or inorganic base selected from the group as described above.

In one embodiment, the reaction in step-(a) is carried out at a temperature of about 0° C. to about 100° C. for at least 30 minutes, specifically at a temperature of about 0° C. to about 50° C. for about 45 minutes to about 5 hours, and most specifically at a temperature of about 0° C. to about 35° C. for about 1 hour to about 4 hours.

The reaction mass containing the compound of formula IV(i) obtained in step-(a) may be subjected to usual work by the methods described herein above. The reaction mass may be used directly in the next step to produce 1,2-dihydro-4-hydroxy-1-methyl-2-oxo-quinolinecarboxylic acid methyl ester of formula III(i), or the compound of formula IV(i) may be isolated by the methods described herein above and then used in the next step.

In one embodiment, the reaction in step-(b) is carried out at a temperature of below about 110° C. for at least 15 minutes, specifically at a temperature of about 50° C. to about 100° C. for about 15 minutes to about 5 hours, and most specifically at a temperature of about 70° C. to about 90° C. for about 30 minutes to about 2 hours.

The reaction mass containing the compound of formula III(i) obtained in step-(b) may be subjected to usual work up by the methods described herein above. The reaction mass may be used directly in the next step to produce 1,2-dihydro-4-hydroxy-1-methyl-2-oxo-quinolinecarboxylic acid of formula II(i), or the compound of formula III(i) may be isolated by the methods described herein above and then used in the next step.

Exemplary third solvents used in step-(c) include, but are not limited to, water, acetic acid, an alcohol, a ketone, an ester, acetonitrile, tetrahydrofuran, dimethylformamide, dimethylsulfoxide, dioxane, and mixtures thereof.

Specifically, the third solvent is selected from the group consisting of water, acetic acid, methanol, ethanol, isopropyl alcohol, propanol, tert-butanol, n-butanol, acetone, methyl ethyl ketone, methyl isobutyl ketone, diethyl ketone, ethyl acetate, methyl acetate, isopropyl acetate, tert-butyl methyl acetate, ethyl formate, acetonitrile, tetrahydrofuran, dimethylformamide, dimethylsulfoxide, dioxane, and mixtures thereof; and more specifically, the third solvent is selected from the group consisting of acetic acid, water, methanol, n-butanol, and mixtures thereof.

In one embodiment, the acid used in step-(c) is selected from the group consisting of hydrochloric acid, hydrobromic acid, hydroiodic acid, acetic acid, oxalic acid, fumaric acid, maleic acid, tartaric acid, di-p-toluoyl-L-(+)-tartaric acid, succinic acid, benzenesulfonic acid, toluenesulfonic acid, methanesulfonic acid, and a combination comprising one or more of the foregoing acids; and a most specific acid is hydrochloric acid.

The hydrochloric acid used may be in the form as described above.

In another embodiment, the reaction in step-(c) is carried out at a temperature of below about 110° C. for at least 30 minutes, specifically at a temperature of about 50° C. to about 100° C. for about 1 hour to about 6 hours, and most specifically at a temperature of about 70° C. to about 90° C. for about 2 hours to about 4 hours.

The reaction mass containing the compound of formula II(i) obtained in step-(c) may be subjected to usual work up by the methods as described above. The reaction mass may be used directly in the next step to produce deschloro laquinimod of formula I(i), or the compound of formula II(i) may be isolated by the methods as described above and then used in the next step.

The coupling agents used in step-(d) is selected from the group as described above. Most specific coupling agents are dichlorotriphenyl phosphorane and thionyl chloride.

Exemplary fourth solvents used in step-(d) include, but are not limited to, a hydrocarbon, a chlorinated hydrocarbon, a ketone, a polar aprotic solvent, an ether, a nitrile, an ester, and mixtures thereof.

Specifically, the fourth solvent is selected from the group consisting of pentane, hexane, heptane, cyclohexane, toluene, xylene, methylene chloride, ethyl dichloride, chloroform, carbon tetrachloride, acetone, methyl isobutyl ketone, N,N-dimethylformamide, N,N-dimethylacetamide, dimethylsulfoxide, diisopropyl ether, methyl tert-butyl ether, tetrahydrofuran, dioxane, acetonitrile, ethyl acetate, isopropyl acetate, and mixtures thereof; and most specifically, the fourth solvent is selected from the group consisting of hexane, heptane, cyclohexane, toluene, methylene chloride, and mixtures thereof.

In one embodiment, the reaction in step-(d) is carried out at a temperature of 0° C. to the reflux temperature of the solvent used for at least 20 minutes, specifically at a temperature of about 0° C. to about 40° C. for about 1 hour to about 20 hours, and most specifically at a temperature of about 0° C. to about 10° C. for about 2 hours to about 7 hours.

In another embodiment, the reaction in step-(d) is carried out in the presence or absence of a base. The base can be an organic or inorganic base selected from the group as described above. A most specific base is diisopropylethylamine.

In another embodiment, the reaction mass containing deschloro laquinimod of formula I(i) obtained in step-(d) may be subjected to usual work up by the methods described herein above.

The isolation of deschloro laquinimod in step-(e) is carried out by forcible or spontaneous crystallization methods described above.

In one embodiment, the crystallization is carried out by cooling the solution while stirring at a temperature of below 25° C., and specifically at about 0° C. to about 20° C.

The recovering in step-(e) is accomplished by the techniques described hereinabove, and the deschloro laquinimod obtained is further dried by the methods described above.

The use of deschloro laquinimod as a reference marker for determining the presence of deschloro laquinimod in laquinimod acid or a salt is by the process comprising: (a) determining the retention time by a column chromatographic method, such as HPLC or TLC, corresponding to the deschloro laquinimod in a reference marker comprising deschloro laquinimod impurity; (b) running a sample of laquinimod acid or a salt on a column chromatography method; and (c) using the retention time in step (a) to identify the presence of deschloro laquinimod impurity in the sample.

In another embodiment, a method is provided for determining the amount of deschloro laquinimod impurity in laquinimod acid or a salt comprising: (a) using a chromatographic method such as HPLC or TLC to measure the area under a peak corresponding to deschloro laquinimod impurity in a reference standard comprising a known amount of deschloro laquinimod impurity; and (b) determining the level of deschloro laquinimod impurity in the sample by comparing the area of step (a) to the area under the peak in a sample comprising a laquinimod acid or a salt contaminated with deschloro laquinimod impurity. A preferable salt of laquinimod is laquinimod sodium.

Further encompassed herein is the use of the highly pure laquinimod or a pharmaceutically acceptable salt thereof having deschloro laquinimod impurity in an amount of about 0.01 area-% to about 0.2 area-%, specifically in an amount of about 0.01 area-% to about 0.15 area-%, for the manufacture of a pharmaceutical composition together with a pharmaceutically acceptable carrier.

A specific pharmaceutical composition of highly pure laquinimod or a pharmaceutically acceptable salt thereof having deschloro laquinimod impurity in an amount of about 0.01 area-% to about 0.2 area-% is selected from a solid dosage form and an oral suspension.

In one embodiment, the highly pure laquinimod or a pharmaceutically acceptable salt thereof having the deschloro laquinimod impurity in an amount of about 0.01 area-% to about 0.2 area-% has a D₉₀ particle size of less than or equal to about 400 microns, specifically less than or equal to about 300 microns, more specifically less than or equal to about 100 microns, still more specifically less than or equal to about 60 microns, and most specifically less than or equal to about 15 microns.

In another embodiment, the particle sizes of the highly pure laquinimod or a pharmaceutically acceptable salt thereof having deschloro laquinimod impurity in an amount of about 0.01 area-% to about 0.2 area-% are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from diseases caused by autoimmunity such as multiple sclerosis, insulin-dependent diabetes mellitus, systemic lupus erythematosus, rheumatoid arthritis, inflammatory bowel disease and psoriasis and, furthermore, diseases where pathologic inflammation plays a major role, such as asthma, atherosclerosis, stroke and Alzheimer's disease, comprising administering a therapeutically effective amount of the highly pure laquinimod or a pharmaceutically acceptable salt thereof having deschloro laquinimod impurity in an amount of about 0.01 area-% to about 0.15 area-%, or a pharmaceutical composition that comprises a therapeutically effective amount of highly pure laquinimod or a pharmaceutically acceptable salt thereof having deschloro laquinimod impurity in an amount of about 0.01 area-% to about 0.15 area-%, along with pharmaceutically acceptable excipients.

According to another aspect, there is provided pharmaceutical compositions comprising highly pure laquinimod or a pharmaceutically acceptable salt thereof having deschloro laquinimod impurity in an amount of about 0.01 area-% to about 0.15 area-% prepared according to the processes disclosed herein and one or more pharmaceutically acceptable excipients.

According to another aspect, there is provided a process for preparing a pharmaceutical formulation comprising combining highly pure laquinimod or a pharmaceutically acceptable salt thereof having deschloro laquinimod impurity in an amount of about 0.01 area-% to about 0.15 area-% prepared according to processes disclosed herein, with one or more pharmaceutically acceptable excipients.

According to another aspect, there is provided pharmaceutical compositions comprising highly pure laquinimod or a pharmaceutically acceptable salt thereof substantially free of at least one, or more, of the deschloro laquinimod, phosphine oxide and quinolinecarboxylic acid impurities prepared according to the processes disclosed herein and one or more pharmaceutically acceptable excipients.

Yet in another embodiment, pharmaceutical compositions comprise at least a therapeutically effective amount of highly pure laquinimod or a pharmaceutically acceptable salt thereof having deschloro laquinimod impurity in an amount of about 0.01 area-% to about 0.15 area-%. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure laquinimod or a pharmaceutically acceptable salt thereof having deschloro laquinimod impurity in an amount of about 0.01 area-% to about 0.15 area-% may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain highly pure laquinimod or a pharmaceutically acceptable salt thereof having deschloro laquinimod impurity in an amount of about 0.01 area-% to about 0.15 area-% within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Experimental:

### HPLC Method for Measuring Chemical Purity:

Related Substances by HPLC (% w/w): (Impurity-A, B, C, D and E)

Water's HPLC system having alliance 2695 model pump and 2487 (UV) detector with Empower chromatography software or its equivalent.

### Chromatographic Parameters:

Column: Hypersil, BDS, C18 (150×4.6 mm, 5μ)
   Make: Thermo scientific, Part No. 28105-154630
Detector: UV at 240 nm,
Flow rate: 1.0 mL/min
Injection volume: 10.0
Run time: 53 min
Oven temperature: 30° C.
Diluent: Water: Acetonitrile (10:90 v/v)

Buffer Preparation:

About 2 mL of triethylamine was added to 1000 mL of water and adjusted the pH to 2.2±0.01 with dilute orthophosphoric acid and followed by filtration through 0.22 μm or finer porosity membrane and degassing.

Mobile Phase-A: Buffer (100%)

Mobile Phase-B: Methanol:Acetonitrile (50:50 v/v)

Gradient Programme:

| | | (%) Mobile |
|---|---|---|
| Time (min) | (%) Mobile phase-A | phase-B |
| 0 | 60 | 40 |
| 3 | 60 | 40 |
| 30 | 25 | 75 |
| 40 | 25 | 75 |
| 42 | 60 | 40 |
| 53 | 60 | 40 |

Sample Preparation:

About 25 mg of sample was weighed and transferred into a 50 mL volumetric flask, dissolved in diluent and made up to the volume with diluent.

The following examples are given for the purpose of illustrating the present disclosure and should not be considered as limitation on the scope or spirit of the disclosure.

### EXAMPLES

### Example 1

### Preparation of N-Ethyl-N-phenyl-1,2-dihydro-4-hydroxy-1-methyl-2-oxoquinoline-3-carboxamide (Deschloro laquinimod impurity)

### Step-I: Preparation of N-methylisatoic anhydride

Isatoic anhydride (50 gm) and anhydrous powdered potassium carbonate (42.29 gm) were mixed with N,N-dimethylformamide (500 ml) at 25-30° C. The reaction mixture was cooled to 0-5° C. followed by slow addition of dimethyl sulfate (42.13 gm) at 0-5° C. The reaction mixture was stirred for 1 hour at 25-30° C. and the completion of the reaction was checked by

TLC. The reaction mass was cooled to 0-5° C., water (500 ml) was added slowly followed by dilute hydrochloric acid at below 15° C. The creamish colored solid was filtered, washed with water and then dried to get N-methylisatoic anhydride (42 gm).

### Step-II: Preparation of 1,2-dihydro-4-hydroxy-1-methyl-2-oxo-3-quinoline carboxylic acid methyl ester

Dimethyl malonate (24.6 gm) and sodium methoxide powder were mixed in N,N-dimethylformamide (225 ml) at 25-30° C. under atmosphere of nitrogen. The mixture was heated at 85° C. and then N,N-dimethylformamide (30 ml) was distilled off under reduced pressure. Methylisatoic anhydride (30 gm) was slowly added to the resulting mass at 80-85° C. under nitrogen atmosphere and the reaction mixture was stirred at 80-85° C. for 30 minutes. After completion of reaction, the mass was cooled to 25-30° C., water (645 ml) was added to the mass followed by the addition of dilute hydrochloric acid (60 ml) to adjust pH to 1. The resulting solid was filtered and washed with water and dried to yield 1,2-dihydro-4-hydroxy-1-methyl-2-oxo-3-quinolinecarboxylic acid methyl ester (30.7 gm).

### Step-III: Preparation of 1,2-dihydro-4-hydroxy-1-methyl-2-oxo-3-quiniline carboxylic acid

1,2-Dihydro-4-hydroxy-1-methyl-2-oxo-3-quinolinecarboxylic acid methyl ester (28 gm) was added to glacial acetic acid (224 ml) at 25-30° C. followed by purging the dry hydrochloride gas and then heated to 80-85° C. The reaction mixture was stirred at 80-85° C. for 2 hours under purging. The resulting mass was cooled to 65° C. followed by the addition of methanol (84 ml) and further cooled to 10-15° C. The resulting solid was filtered and washed with methanol (28 ml) and then dried to get 1,2-dihydro-4-hydroxy-1-methyl-2-oxo-3-quinolinecarboxylic acid (25 gm).

### Step-IV: Preparation of Deschloro Laquinimod

The mixture of 1,2-Dihydro-4-hydroxy-1-methyl-2-oxo-3-quinolinecarboxylic acid (5 gm) and methylene chloride (200 ml) were cooled to 0-5° C., diisopropylethyl amine (10.3 gm) was added to the resulting mixture at 0-5° C. followed by the addition of N-ethyl aniline (3.04 gm) at 0-5° C. A solution of thionyl chloride (3.53 gm) dissolved in methylene chloride (10 ml) was added to the reaction mixture at 0-5° C. and then stirred for 3 hours at 0-5° C. After completion of the reaction, methylene chloride was distilled out under vacuum at below 40° C. The resulting residue was treated with ethyl acetate and 10% sodium hydroxide solution (100 ml). The two layers were separated and washed the aqueous layer with ethyl acetate (30 ml). The aqueous layer was acidified with dilute hydrochloric acid at 10-15° C. The resulting solid obtained was filtered, washed with water and dried to get deschloro laquinimod (5.4 gm).

### Example 2

### Preparation of Triphenylphosphine oxide (phosphine oxide impurity)

Dichlorotriphenyl phosphorane (10 gm) was added to water (100 ml) at 25-30° C. and then stirred the white colored suspension at 25-30° C. for 2 hours. The white colored solid was filtered and dried to get triphenylphosphine oxide (5 gm).

### Example 3

### Preparation of Pure N-Ethyl-N-phenyl-1,2-dihydro-4-hydroxy-5-chloro-1-methyl-2-oxoquinoline-3-carboxamide sodium salt (Laquinimod Sodium)

### Step-I: Preparation of 5-Chloro-N-methylisatoic anhydride

The mixture of 5-chloroisatoic anhydride (200 gm; content of isatoic anhydride impurity: 1.4%), powdered sodium carbonate (139.74 gm) and N,N-dimethylformamide (200 ml) was stirred at 25-30° C. under nitrogen atmosphere for 10 minutes. The reaction mixture was cooled to 0-5° C. under nitrogen atmosphere followed by the addition of dimethyl sulfate (139.22 gm) and then stirred for 15 minutes at 0-5° C. The resulting mass was heated to 25-30° C. and stirred for 60 minutes at 25-30° C. This was followed by slow addition of 1N hydrochloride acid (200 ml) at 5-10° C. for 45 minutes and the resulting suspension was stirred at 5-10° C. for 1 hour. The solid was filtered and washed with water (2000 ml) and then dried at 55-60° C. to yield 180 g of 5-chloro-N-methylisatoic anhydride (HPLC purity: 98%, Content of N-methylisatoic anhydride: 0.6%).

### Step-II: Preparation of 5-Chloro-1,2-dihydro-4-hydroxy-1-methyl-2-oxo-3-quinoline carboxylic acid methyl ester

Dimethyl malonate (123.57 gm) and sodium methoxide (68.01 gm) were added to N,N-dimethyl formamide (1350 ml) at 25-30° C. under nitrogen atmosphere, the reaction mixture was heated to 80-85° C. and stirred at 80-85° C. for 1 hour 30 minutes. N,N-Dimethylformamide (190 ml) was distilled off under reduced pressure (50 mbar), the temperature of the resulting mass was adjusted to 80° C. and then 5-chloro-N-methylisatoic anhydride (180 gm) was added carefully during 10 minutes. The reaction mixture was stirred for 1 hour at 80-85° C. and the resulting mass was cooled to 25-30° C. followed by the addition of water (3870 ml) for 1 hour at 25-30° C. The pH of the resulting mass was adjusted to 1 by using 1 N hydrochloric acid at 25-30° C. and the suspension was stirred for 2 hours at 25-30° C. The resulting solid was filtered and washed with water (2700 ml) and then dried at 55-60° C. until constant weight to give 190 g of 5-Chloro-1,2-dihydro-4-hydroxy-1-methyl-2-oxo-3-quinolinecarboxylic acid methyl ester (HPLC purity: 97%; Content of 1,2-dihydro-4-hydroxy-1-methyl-2-oxo-3-quinolinecarboxylic acid methyl ester: 0.3%).

### Step-III: Preparation of 5-Chloro-1,2-dihydro-4-hydroxy-1-methyl-2-oxo-3-quinolinecarboxylic acid

The mixture of 5-Chloro-1,2-dihydro-4-hydroxy-1-methyl-2-oxo-3-quinolinecarboxylic acid methyl ester (190 gm) and glacial acetic acid (1520 ml) was heated at 80-85° C. followed by purging dry HCl gas at 80-85° C. until the percentage of 5-Chloro-1,2-dihydro-4-hydroxy-1-methyl-2-oxo-3-quinolinecarboxylic acid methyl ester is less than 0.1% by HPLC. The suspension was cooled to 65° C. and then methanol (570 ml) was added at 60-65° C. The resulting suspension was again cooled to 25-30° C. and stirred for 1 hour at 25-30° C. The resulting solid was filtered and washed with methanol (190 ml) and then dried the solid at 55-60° C. until constant weight to give 145 g of 5-Chloro-1,2-dihydro-4-hydroxy-1-methyl-2-oxo-3-quinolinecarboxylic acid (HPLC purity: 99%; Content of 1,2-dihydro-4-hydroxy-1-methyl-2-oxo-3-quinolinecarboxylic acid: 0.07%).

### Step-IV: Preparation of crude Laquinimod

N-Ethyl aniline (5.2 gm) was added to methylene dichloride (100 ml) and then stirred for 10 minutes at 25-30° C. under nitrogen atmosphere. This was followed by the addition of dichlorotriphenyl phosphorane (26.6 gm) at 25-30° C. and the reaction mass was stirred for 15 minutes. This was followed by the addition of 5-chloro-1,2-dihydro-4-hydroxy-1-methyl-2-oxo-3-quinolinecarboxylic acid (10 gm) at 25-30° C., the reaction mixture was heated to reflux (40-45° C.) and maintained for 3 hours. The resulting mass was cooled to 25-30° C. followed by the addition of water (100 ml) at 25-30° C., the resulting biphasic layer was stirred at 25-30° C. for 15 minutes and then separated the two layers. The organic layer was washed with water (100 ml) at 25-30° C. and methylene dichloride was distilled out completely under vacuum at below 40° C. to give 15 gm of crude laquinimod as residue [HPLC Purity: 97%; Content of Impurities: Deschloro laquinimod impurity (Impurity-B): 0.05%; Triphenylphosphine oxide (Impurity-C): 0.1%; Quinolinecarboxylic acid impurity (Impurity-D): 1%].

### Step-V: Purification of Laquinimod

Methylene dichloride (10 ml) and ethyl acetate (100 ml) were added to the residue (obtained in step-IV) at 40-45° C., the resulting suspension was heated to reflux and stirred for 15 minutes at reflux. The white colored suspension was cooled at 25-30° C. and stirred for 1 hour at 25-30° C. The resulting solid was filtered and washed with ethyl acetate (2×200 ml). The white colored solid was dried to constant weight to yield 11.7 g of pure laquinimod [HPLC purity: 99.47%; Content of Impurities: Deschloro laquinimod impurity (Impurity-B): 0.04%; Triphenylphosphine oxide (Impurity-C): 0.03%; Quinolinecarboxylic acid impurity (Impurity-D): 0.2%].

### Step-VI: Preparation of Pure Laquinimod Sodium

Laquinimod (10 gm, obtained in step-V) was stirred with absolute ethanol (40 ml) for 30 minutes at 25-30° C. followed by the addition of sodium hydroxide solution (1.18 gm dissolved in 3 ml water) at 25-30° C. The resulting clear solution was stirred for 30 min at 25-30° C. followed by filtration through hyflow bed and washed the bed with ethanol (30 ml). The clear filtrate was seeded with pure laquinimod sodium and stirred at 25-30° C. for 3 hours. The resulting white colored suspension was cooled to 0-5° C. and stirred for 2 hours at 0-5° C. The solid was filtered and washed with chilled absolute ethanol (10 ml) and then the white solid was dried at 60-65° C. to constant weight to yield 8 gm of N-ethyl-N-phenyl-1,2-dihydro-4-hydroxy-5-chloro-1-methyl-2-oxo-quinoline sodium salt (Laquinimod Sodium) [HPLC purity: 99.92%; Content of Impurities: Deschloro laquinimod impurity (Impurity-B): 0.04%; Triphenylphosphine oxide (Impurity-C): 0.01%; Quinolinecarboxylic acid impurity (Impurity-D): 0.01%].

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP).

The term “crude laquinimod or a pharmaceutically acceptable salt thereof” as used herein refers to laquinimod or a pharmaceutically acceptable salt thereof containing greater than about 0.2 area-%, more specifically greater than about 0.25 area-%, still more specifically greater than about 0.4 area-% and most specifically greater than about 1 area-% of at least one, or more, of the deschloro laquinimod, phosphine oxide and quinolinecarboxylic acid impurities (measured by HPLC).

As used herein, the term, “detectable” refers to a measurable quantity measured using an HPLC method having a detection limit of 0.01 area-%.

As used herein, in connection with amount of impurities in laquinimod or a pharmaceutically acceptable salt thereof, the term “not detectable” means not detected by the herein described HPLC method having a detection limit for impurities of 0.01 area-%.

As used herein, “limit of detection (LOD)” refers to the lowest concentration of analyte that can be clearly detected above the base line signal, is estimated is three times the signal to noise ratio.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent. “Mean particle size distribution, i.e., (D₅₀)” correspondingly, means the median of said particle size distribution.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Laquinimod or a pharmaceutically acceptable salt thereof comprising a N-ethyl-N-phenyl-1,2-dihydro-4-hydroxy-1-methyl-2-oxoquinoline-3-carboxamide (deschloro laquinimod impurity) in an amount of about 0.01 area-% to about 0.2 area-% as measured by HPLC, wherein the laquinimod has a purity of about 99% to about 99.99% as measured by HPLC.

2. (canceled)

3. (canceled)

4. Laquinimod of claim 1, further comprising one, or both, of a phosphine oxide and a quinolinecarboxylic acid impurities, each one, in an amount of less than about 0.2 area-% as measured by HPLC; wherein the phosphine oxide impurity is triphenylphosphine oxide, and the quinolinecarboxylic acid impurity is 5-chloro-1,2-dihydro-4-hydroxy-1-methyl-2-oxo-quinolinecarboxylic acid; and wherein the pharmaceutically acceptable salt of laquinimod is an alkali or alkaline earth metal salt.

5. (canceled)

6. Laquinimod of claim 4, having a non-detectable amount of one, or both, of the phosphine oxide and the quinolinecarboxylic acid impurities as measured by HPLC; and wherein the pharmaceutically acceptable salt of laquinimod is laquinimod sodium.

7. (canceled)

8. (canceled)

9. An isolated deschloro laquinimod, N-ethyl-N-phenyl-1,2-dihydro-4-hydroxy-1-methyl-2-oxoquinoline-3-carboxamide, of formula I(i): 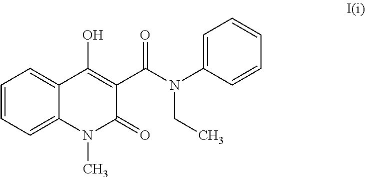
or a pharmaceutically acceptable salt thereof.

10. A process for preparing the highly pure laquinimod or a pharmaceutically acceptable salt thereof of claim 1, comprising:
a) methylating 5-chloroisatoic anhydride of formula V: 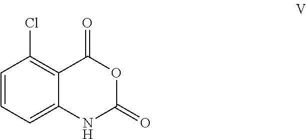
containing isatoic anhydride impurity of formula V(i) in an amount of less than about 1.5 area-% (measured by HPLC), with dimethylsulfate in the presence of a base in a first solvent to provide 5-chloro-N-methylisatoic anhydride of formula IV: 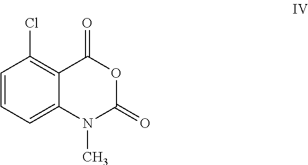
containing N-methylisatoic anhydride impurity of formula IV(i) in an amount of less than about 0.8 area-%;
b) reacting the compound obtained in step-(a) with dimethyl malonate in the presence of sodium methoxide in a second solvent to provide 5-chloro-1,2-dihydro-4-hydroxy-1-methyl-2-oxo-quinolinecarboxylic acid methyl ester of formula III: 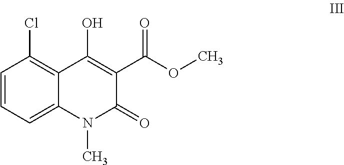
containing 1,2-dihydro-4-hydroxy-1-methyl-2-oxo-quinolinecarboxylic acid methyl ester impurity of formula III(i) in an amount of less than about 0.5 area-%;
c) hydrolyzing the ester compound obtained in step-(b) by treatment with hydrochloric acid in the presence of acetic acid to provide 5-chloro-1,2-dihydro-4-hydroxy-1-methyl-2-oxo-quinoline carboxylic acid of formula II: 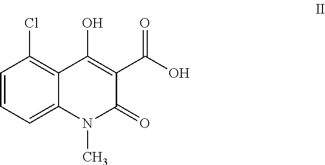
containing 1,2-dihydro-4-hydroxy-1-methyl-2-oxo-quinoline carboxylic acid impurity of formula II(i) in an amount of about 0.01 area-% to about 0.2 area-%;
d) condensing the quinolinecarboxylic acid compound obtained in step-(c) with N-ethylaniline in the presence of a coupling agent in a third solvent to provide a reaction mass containing N-ethyl-N-phenyl-1,2-dihydro-4-hydroxy-5-chloro-1-methyl-2-oxoquinoline-3-carboxamide (laquinimod) of formula I containing N-ethyl-N-phenyl-1,2-dihydro-4-hydroxy-1-methyl-2-oxoquinoline-3-carboxamide impurity (deschloro laquinimod impurity) of formula I(i); and
e) isolating and/or recovering highly pure laquinimod of formula I containing deschloro laquinimod impurity (impurity-B) of formula I(i) in an amount of about 0.01 area-% to about 0.2 area-% as measured by HPLC from the reaction mass obtained in step-(d), and optionally converting the laquinimod obtained into a pharmaceutically acceptable salt thereof.

11. The process of claim 10, wherein the first and second solvents used in steps-(a) and (b) are, each independently, selected from the group consisting of N,N-dimethylformamide, N,N-dimethylacetamide, dimethylsulfoxide, tetrahydrofuran, dioxane, and mixtures thereof; wherein the third solvent used in step-(d) is selected from the group consisting of pentane, hexane, heptane, cyclohexane, toluene, xylene, methylene chloride, ethyl dichloride, chloroform, carbon tetrachloride, acetone, methyl isobutyl ketone, N,N-dimethylformamide, N,N-dimethylacetamide, dimethylsulfoxide, diisopropyl ether, methyl tert-butyl ether, tetrahydrofuran, dioxane, acetonitrile, ethyl acetate, isopropyl acetate, and mixtures thereof; and wherein the base used in step-(a) is an organic or inorganic base selected from the group consisting of triethylamine, tributylamine, diisopropylethylamine, diethylamine, tert-butylamine, N-methylmorpholine, pyridine, 4-(N,N-dimethylamino)pyridine, ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide, potassium tert-butoxide, and mixtures thereof.

12. The process of claim 11, wherein the first solvent is N,N-dimethylformamide; wherein the second solvent is N,N-dimethylformamide; and wherein the third solvent is selected from the group consisting of hexane, heptane, cyclohexane, toluene, methylene chloride, and mixtures thereof.

13. (canceled)

14. The process of claim 10, wherein the reaction in step-(a) is carried out at a temperature of about 0° C. to about 100° C.; wherein the reaction in step-(b) is carried out at a temperature of about 50° C. to about 100° C.; wherein the reaction in step-(c) is carried out at a temperature of below about 110° C.; wherein the reaction in step-(d) is carried out at a temperature of 0° C. to the reflux temperature of the solvent used; and wherein the isolation of highly pure laquinimod in step-(e) is carried out by forcible or spontaneous crystallization, wherein the forcible crystallization is initiated by cooling, seeding, partial removal of the solvent from the solution, by combining an anti-solvent with the solution, or a combination thereof.

15. The process of claim 10, wherein the coupling agent used in step-(d) is selected from the group consisting of N,N′-carbonyldiimidazole (CDI), N,N′-diethylcarbodiimide, N,N′-dipropylcarbodiimide, N,N′-diisopropylcarbodiimide, N,N′-dicyclohexylcarbodiimide, N-ethyl-N′-(3-dimethylamino propyl)-carbodiimide, thionyl chloride, dichlorotriphenyl phosphorane, phosphorous oxychloride, O-benzotriazole-N,N,N′,N′-tetramethyl-uronium-hexafluoro-phosphate (HBTU), and combinations comprising one or more of the foregoing coupling agents; and wherein the reaction in step-(d) is carried out in the presence or absence of a base, wherein the base is selected from the group consisting of triethylamine, tributylamine, diisopropylethylamine, diethylamine, tert-butylamine, N-methylmorpholine, pyridine, 4-(N,N-dimethylamino) pyridine, ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide, potassium tert-butoxide, and mixtures thereof.

16. The process of claim 15, wherein the coupling agent is dichlorotriphenyl phosphorane.

17. (canceled)

18. (canceled)

19. (canceled)

20. (canceled)

21. A process for preparing the highly pure laquinimod or a pharmaceutically acceptable salt thereof of claim 1, comprising:
a) providing a suspension of crude laquinimod in a solvent medium comprising an ester solvent and a chlorinated hydrocarbon solvent;
b) optionally, cooling the suspension obtained in step-(a); and
c) recovering highly pure laquinimod substantially free of impurities from the suspension and optionally converting the highly pure laquinimod obtained into a pharmaceutically acceptable salt thereof.

22. The process of claim 21, wherein the halogenated hydrocarbon solvent used in step-(a) is selected from the group consisting of methylene chloride, 1,2-dichloroethane, chloroform, carbon tetrachloride, and mixtures thereof; wherein the ester solvent is selected from the group consisting of methyl acetate, ethyl acetate, n-propyl acetate, isopropyl acetate, n-butyl acetate, isobutyl acetate, tert-butyl acetate, ethyl formate, and mixtures thereof; wherein the suspension in step-(a) is provided by suspending crude laquinimod in the solvent medium while stiffing at below reflux temperature of the solvent medium used; wherein the suspension in step-(b) is cooled at a temperature of about 10° C. to about 30° C. for about 30 minutes to 2 hours; and wherein the recovering in step-(c) is carried out by filtration, filtration under vacuum, decantation, centrifugation, filtration employing a filtration media of a silica gel or celite, or a combination thereof.

23. The process of claim 22, wherein the halogenated hydrocarbon solvent methylene chloride; wherein the ester solvent is ethyl acetate, isopropyl acetate, or a mixture thereof; and wherein the suspension in step-(a) is stirred at a temperature of about 30° C. to about 100° C.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The highly pure laquinimod or a pharmaceutically acceptable salt thereof of claim 1, further comprising one or more pharmaceutically acceptable excipients to form a pharmaceutical composition.

38. (canceled)

39. (canceled)

40. (canceled)

41. (canceled)

42. (canceled)

43. The pharmaceutical composition of claim 37, wherein the highly pure laquinimod or a pharmaceutically acceptable salt thereof has a D₉₀ particle size of less than or equal to about 400 microns.

44. The pharmaceutical composition of claim 43, wherein the D₉₀ particle size is less than or equal to about 300 microns; less than or equal to about 100 microns; less than or equal to about 60 microns; or less than or equal to about 15 microns.

45. (canceled)

46. (canceled)

47. (canceled)
